(19) United States
(12) Patent Application Publication
(10) Pub. No.: US 2022/0254487 A1
(43) Pub. Date: Aug. 11, 2022

(54) Method and System for Indirect Contamination Tracking

(71) Applicant: Dell Products L.P., Round Rock, TX (US)

(72) Inventors: Michiel Sebastiaan Emanuel Petrus Knoppert, Amsterdam (NL); Gerald Rene Pelissier, Mendham, NJ (US); Thomas Marcus Hinskens, Utrecht (NL)

(73) Assignee: Dell Products L.P., Round Rock, TX (US)

(21) Appl. No.: 17/171,970

(22) Filed: Feb. 9, 2021

(51) Int. Cl. G16H 40/67; G16H 50/80

(52) U.S. Cl. CPC G16H 40/67; G16H 50/80

(57) ABSTRACT

A system, method, and computer-readable medium are disclosed for managing indirect contamination of devices in a defined environment. A user device associated with a particular user is connected with one more devices that are shared with other users in a defined environment. Proximity data of the user device and the one or more shared devices is provided to an administrative service of the defined environment. If the user device is determined to be contaminated, the user device is flagged, and notification is provided to the other users of the shared devices as to possible contamination risk.

### BACKGROUND OF THE INVENTION

### Field of the Invention

The present invention relates to tracking of information handling systems and devices. More specifically, embodiments of the invention relate to management as to indirect contamination of information handling systems and devices in a defined environment.

### Description of the Related Art

Infectious diseases, such as those spread by viruses, can be tracked using tools such as tracker applications on personal mobile devices (i.e., smart phones). Such mobile devices acting as proxies for their users. In other words, the mobile device is tracked or associated with the user. Therefore, if a particular mobile device associated with a user, becomes in contact with another mobile device associated with an infected user, a notification can be made to the particular mobile device and its user of a contamination risk.

Such notifications through communication between mobile devices may not alert contamination risks as to surfaces or other devices that may have been in contact with an infected user. In other words, contamination through indirect transmission. If an infected user comes into contact with a surface or another mobile device that is not implemented with a tracker applications, contamination notification and alerts may not be provided.

Viruses and other transmittable organisms can easily be spread in household and office environments, particularly on surfaces of commonly used and shared appliances and devices, such as headsets, keyboards, desks, etc. Therefore, there is a need to be able to track the potential spread of indirect transmission by infected users in a such environments.

### SUMMARY OF THE INVENTION

A system, method, and computer-readable medium are disclosed for managing indirect contamination of devices in a defined environment. A user device associated with a particular user is connected with one more devices that are shared with other users in a defined environment. Proximity data of the user device and the one or more shared devices is provided to an administrative service of the defined environment. If the user device is determined to be contaminated, the user device is flagged, and notification is provided to the other users of the shared devices as to possible contamination risk.

### BRIEF DESCRIPTION OF THE DRAWINGS

The use of the same reference number throughout the several figures designates a like or similar element. The present invention may be better understood, and its numerous objects, features, and advantages made apparent to those skilled in the art by referencing the accompanying drawings, wherein:

FIG. 1 depicts a general illustration of a computing system as implemented in the system and method of the present invention;

FIG. 2 depicts a system for system for management as to indirect contamination tracking in a defined environment;

FIG. 3 shows a flow chart example scenario of indirect contamination tracking that implements a system for indirect contamination tracking in a defined environment;

FIG. 4 shows a flow chart for management as to indirect contamination of information handling systems and devices in a defined environment.

### DETAILED DESCRIPTION

A system, method, and computer readable medium are disclosed for indirect contamination tracking in a defined environment through an administrative service using Bluetooth enabled devices that provide proximity data within the defined environment to as to contamination risks.

In certain implementations, an administrative service manages the defined environment's indirect contamination risks. The defined environment can be an office building, a home, laboratory, campus, etc.

Implementations provide for the Bluetooth enabled user devices and shared devices to record and send proximity data of user devices to the administrative service. The administrative service logs received proximity data to a database, where the logged proximity data can be shared with a public health data base, such as a government health agency infectious disease data base.

Certain implementations provide that when a new infection is registered at the public health data base, the administrative service is notified of possible device exposures, such as for a certain day. With the new infection data, the administrative service can establish additional indirect points of contact. For example, movement of a user device can be tracked back as to movements within the defined environment and checking log in data of affected (i.e., possibly contaminated) shared devices in the defined environment.

Implementations can be provided where the administrative service takes action instructing defined environment facilities, such as information technology (IT) services to perform a cleaning action on affected shared devices and affected areas of the defined environment. Affected shared devices can notify potential users of the shared devices, such as those within a defined proximity range, of contamination risk. When a user is logged on a possible contaminated device, notification can be made as to indirect contamination risk when identification (ID) of the user device is established. An ID may be a Bluetooth ID associated with the user device. Anonymous warning messages can be sent by to users, including shared users, via Bluetooth communication.

For purposes of this disclosure, a device or an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, an information handling system may be a personal computer, a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, and a video display. The information handling system may also include one or more buses operable to transmit communications between the various hardware components.

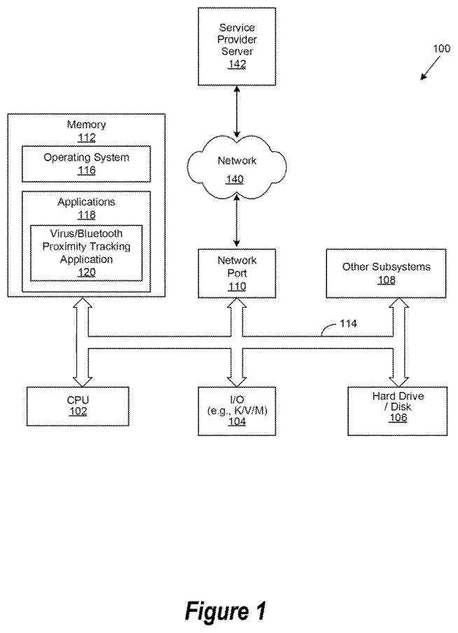

FIG. 1 illustrates an information handling system 100 that can be used to implement the system and method of the present invention. The information handling system 100 can be implemented as a portable device, such as a mobile or smart phone. The information handling system 100 includes one or more processors (e.g., central processor unit or “CPU”, embedded controller, etc.) 102, input/output (I/O) devices 104, such as a display, a keyboard, a mouse, and associated controllers, a hard drive or disk storage 106, and various other subsystems 108.

In various embodiments, the information handling system 100 also includes network port 110 operable to connect to a network 140, which is likewise accessible by a service provider server 142. The network 140 may be a public network, such as the Internet, a physical private network, a wireless network, a virtual private network (VPN), or any combination thereof. Skilled practitioners of the art will recognize that many such embodiments are possible, and the foregoing is not intended to limit the spirit, scope or intent of the invention. Implementations provide for wireless connection of the information handling system 100 to network 140 and other devices using technology such as Bluetooth.

The information handling system 100 likewise includes system memory 112, which is interconnected to the foregoing via one or more buses 114. System memory 112 further includes an operating system (OS) 116 and applications 118. In certain embodiments, applications 118 are provided as a service from the service provider server 142. Implementations can provide for applications 118 to include a virus application 120. Virus/Bluetooth proximity tracking application 120 allows for methods described herein to be implemented. The virus/Bluetooth proximity tracking application 120 is further described below.

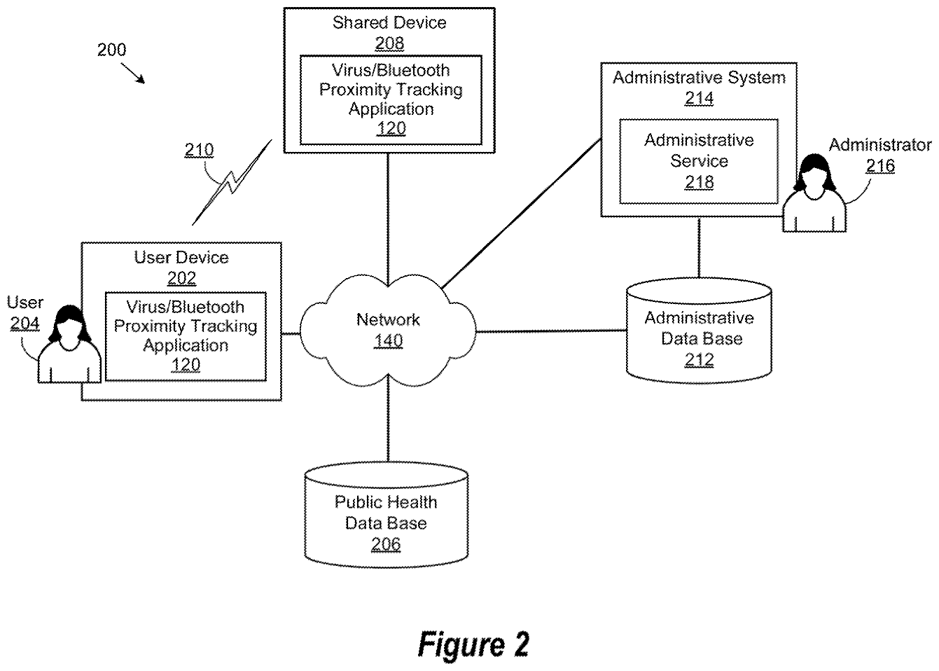

FIG. 2 is a simplified block diagram of a system for management as to indirect contamination tracking in a controlled environment. The system 200 includes a user device 202 that can be configured as the information handing system 100 as described above in reference to FIG. 1. As discussed, user device 202 can be implemented as a portable device, such as mobile or smart phone. The user device is associated with a user 204.

System 200 further includes the network 140. Network 140 represents a various private and public networks and can as discussed can include wired and wireless networks. Implementations provide for the network 140 to include networks that are exclusive to a managed environment, such as an office building, a home, laboratory, campus, or other defined environment. In addition, implementations provide for the network to include public domain networks. As will be evident in the description, the user device 202 in various implementations can have access to various to other devices and/or services through network 140.

Various implementations provide for the user device 202 to include a unique identifier (ID), and in particular a Bluetooth ID. In various implementations, the user 204 is anonymously identified through the Bluetooth ID of the user device 202. For example, communication is anonymously performed with user 204 by identifying the Bluetooth ID of the user device 202. The user device 202 being associated with user 204

Embodiments provide for the system 200 to include a public health data base 206. The public health data base 206 can include governmental health agency data bases. The user device 202 can communicate with the public health data base 204 through public domain networks of network 140. Various implementations provide for public health data base 204 to store proximity data related to the user device 202, which tracks by association the user 204. Therefore, if user 204 becomes infected and gives permission to share infection status, a flag can be set anonymously as to the Bluetooth ID of the user device 202.

Implementations provide for when the user device 202 and user 204 enter a managed defined environment, such as an office building, a home, laboratory, campus, etc., the user device 202 connects with the networks (i.e., network 140) of the managed defined environment. The managed defined environment can include various shared devices, as represented by shared device 208. The shared device can be implemented as an information handling system 100. Examples of shared device 206 include commonly used and shared appliances and devices, such as headsets, computer systems, and can associated connected accessories including keyboards, desks, etc. Shared device 208 can be connected to the networks (i.e., network 140) of the managed defined environment.

Embodiments provide for user device 202 to communicate with shared device 208 via Bluetooth communication 210. Implementations provide for the shared device 206 to include a Bluetooth proximity tracking application 210. The user device 202 is tracked by the shared device 208 using the virus/Bluetooth proximity tracking application 120. In particular proximity data of the user device 202 is obtained. Such proximity data can include contact with and proximity the shared device 208. Therefore, if a user 204 is determined to be infected, the user device 202 of the user 204 can be flagged and identified by its Bluetooth ID.

Embodiments provide for the system 200 to include an administrative data base 212. In various implementations, the proximity data described above is stored in the administrative data base 212. An administrative system 214 can be included in system 200. Administrative system 214 represents various systems, which can include information technology (IT) services and other support services of the managed defined environment. The administrative system 214 accesses and communicates with the administrative data base 212 directly or through network 140. Furthermore, the administrative system 214 accesses and communicates with the public health data base 204. In certain implementations, the administrative system 214 directly communicates with the user device 202 and shared device 208.

Implementations provide for the administrative system 214 include an administrative service 218. In particular, the administrative service 218 can be configured to access and process proximity data for administrative data base 212, provide notifications and alerts to user regarding contamination risks, and communicate and provide proximity data to the public health data base 204. In certain implementations, the administrative service 218 directly receives proximity data and logs proximity data to the administrative data base 212.

Shared device 206 risk exposure that includes day of occurrence can be included in the proximity data. Such exposure and day of occurrence can be communicated to the administrative service 218. In various implementations, the administrative service 218 finds contact points in the administrative data base 212, notifies users (e.g., shared users) that may be at risk, and notifies administrator 218 (e.g., IT personnel, facilities, etc.) to perform a cleaning action on potential infected shared devices.

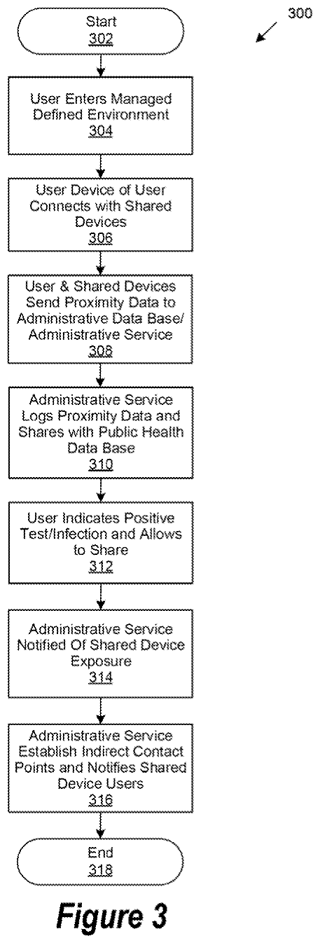

FIG. 3 is an example scenario of indirect contamination tracking that implements system 200. The flowchart 300 illustrates one scenario; however, it is to be understood that other scenarios are possible for which system 200 supports. The order in which the method is described is not intended to be construed as a limitation, and any number of the described method blocks may be combined in any order to implement the method, or alternate method. Additionally, individual blocks may be deleted from the method without departing from the spirit and scope of the subject matter described herein.

At step 302, the process 300 starts. At step 304, user 204 and user device 202 enters a defined environment, such as an office building, a home, laboratory. At step 306, through a connection, such as Bluetooth enabled communication, the user device 202 and shared devices (e.g., shared device 208) of the defined environment connect with one another. At step 308, user device 202 and shared devices (e.g., shared device 208), send proximity data to administrative service 218 and/or administrative data base 212. At step 310, the administrative service 218 logs the proximity data to the administrative data base 212 and shares the proximity data base with public health data base 206. At step 312, if the user 204 tests positive for contamination and allows such information to be shared, the user device 202 is flagged. At step 314, the administrative service 218 is notified of the user contamination. The notification is based on an ID of the user device 202, such as a unique Bluetooth ID given to the user device 202. At step 316, the administrative service 218 establishes indirect contact points and provides notification of contamination risks to users of the shared devices (e.g., shared device 208). At step 318, the process 300 ends.

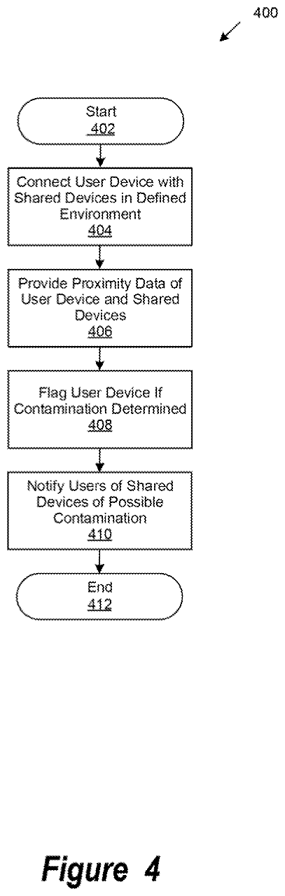

FIG. 4 is a generalized flowchart 400 for management as to indirect contamination of information handling systems and devices in a defined environment. The order in which the method is described is not intended to be construed as a limitation, and any number of the described method blocks may be combined in any order to implement the method, or alternate method. Additionally, individual blocks may be deleted from the method without departing from the spirit and scope of the subject matter described herein. Furthermore, the method may be implemented in any suitable hardware, software, firmware, or a combination thereof, without departing from the scope of the invention.

At step 402, the process 400 starts. At step 404, a connecting is made between a user device and one or more shared devices in a defined environment. The connection can be made through a Bluetooth connection between the user device and the shared devices. The user device is associated with a particular user. The user device and the shared devices can have unique identifiers, such a Bluetooth IDs.

At step 406, proximity data between the user device and the one or more shared devices are provided to an administrative service and/or an administrative data base.

At step 408, if a contamination is determined or associated with the user device, the user device is flagged as contaminated. The particular user may indicate that they have been contaminated and provide an anonymous indication of such through the user device's identifier (e.g., Bluetooth ID).

At step 410, notification is provided to users of the one or more shared devices of possible contamination and contamination risk. The notification can be through unique identifiers (e.g., Bluetooth ID) of the one or more shared user devices. At step 4120, the process 400 ends.

As will be appreciated by one skilled in the art, the present invention can be embodied as a method, system, or computer program product. Accordingly, embodiments of the invention can be implemented entirely in hardware, entirely in software (including firmware, resident software, micro-code, etc.) or in an embodiment combining software and hardware. These various embodiments can all generally be referred to herein as a “circuit,” “module,” or “system.” Furthermore, the present invention can take the form of a computer program product on a computer-usable storage medium having computer-usable program code embodied in the medium.

Any suitable computer usable or computer readable medium can be utilized. The computer-usable or computer-readable medium can be, for example, but not limited to, an electronic, magnetic, optical, electromagnetic, infrared, or semiconductor system, apparatus, or device. More specific examples (a non-exhaustive list) of the computer-readable medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, or a magnetic storage device. In the context of this document, a computer-usable or computer-readable medium can be any medium that can contain, store, communicate, or transport the program for use by or in connection with the instruction execution system, apparatus, or device.

Computer program code for carrying out operations of the present invention can be written in an object oriented programming language such as Java, Smalltalk, C++ or the like. However, the computer program code for carrying out operations of the present invention can also be written in conventional procedural programming languages, such as the “C” programming language or similar programming languages. The program code can execute entirely on the user's computer, partly on the user's computer, as a stand-alone software package, partly on the user's computer and partly on a remote computer or entirely on the remote computer or server. In the latter scenario, the remote computer can be connected to the user's computer through a local area network (LAN) or a wide area network (WAN), or the connection can be made to an external computer (for example, through the Internet using an Internet Service Provider).

Embodiments of the invention are described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions can also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions can also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The present invention is well adapted to attain the advantages mentioned as well as others inherent therein. While the present invention has been depicted, described, and is defined by reference to particular embodiments of the invention, such references do not imply a limitation on the invention, and no such limitation is to be inferred. The invention is capable of considerable modification, alteration, and equivalents in form and function, as will occur to those ordinarily skilled in the pertinent arts. The depicted and described embodiments are examples only and are not exhaustive of the scope of the invention.

Consequently, the invention is intended to be limited only by the spirit and scope of the appended claims, giving full cognizance to equivalents in all respects.

What is claimed is:

1. A computer-implementable method of managing indirect contamination of devices in a defined environment comprising:
connecting a user device with one or more shared devices in the defined environment;
providing proximity data of the user device and the one or more shared devices to an administrative service;
flagging the user device if a determination is made as to contamination associated with the user device; and
notifying users of the shared devices of the one or more shared devices of possible contamination risk.

2. The method of claim 1, wherein the defined environment is an office building, home, campus, or laboratory.

3. The method of claim 1, wherein the user device and the one or more shared devices is assigned a unique Bluetooth identifier and the connecting is performed through Bluetooth communication.

4. The method of claim 1, wherein the flagging is initiated when it is determined that a user of the user device is infected.

5. The method of claim 1, wherein the notifying is performed using unique Bluetooth identifiers assigned to the user device and the one or more shared devices.

6. The method of claim 1 further comprising determining infection registered at a public health data base associated with user device and possible user device exposures over a certain day.

7. The method of claim 1 further comprising logging proximity data received by the administrative service and sharing with a public health data base.

8. A system comprising:
a processor;
a data bus coupled to the processor; and
a non-transitory, computer-readable storage medium embodying computer program code, the non-transitory, computer-readable storage medium being coupled to the data bus, the computer program code interacting with a plurality of computer operations and comprising instructions executable by the processor and configured for:
connecting a user device with one or more shared devices in the defined environment;
providing proximity data of the user device and the one or more shared devices to an administrative service;
flagging the user device if a determination is made as to contamination associated with the user device; and
notifying users of the shared devices of the one or more shared devices of possible contamination risk.

9. The system of claim 8, wherein the defined environment is an office building, home, campus, or laboratory.

10. The system of claim 8, wherein the user device and the one or more shared devices is assigned a unique Bluetooth identifier and the connecting is performed through Bluetooth communication.

11. The system of claim 8, wherein the flagging is initiated when it is determined that a user of the user device is infected.

12. The system of claim 8, wherein the notifying is performed using unique Bluetooth identifiers assigned to the user device and the one or more shared devices.

13. The system of claim 8 further comprising determining infection registered at a public health data base associated with user device and possible user device exposures over a certain day.

14. The system of claim 8 further comprising logging proximity data received by the administrative service and sharing with a public health data base.

15. A non-transitory, computer-readable storage medium embodying computer program code, the computer program code comprising computer executable instructions configured for:
providing proximity data of the user device and the one or more shared devices to an administrative service;
flagging the user device if a determination is made as to contamination associated with the user device; and
notifying users of the shared devices of the one or more shared devices of possible contamination risk.

16. The non-transitory, computer-readable storage medium of claim 15, wherein the user device and the one or more shared devices is assigned a unique Bluetooth identifier and the connecting is performed through Bluetooth communication.

17. The non-transitory, computer-readable storage medium of claim 15, wherein the flagging is initiated when it is determined that a user of the user device is infected.

18. The non-transitory, computer-readable storage medium of claim 15, wherein the notifying is performed using unique Bluetooth identifiers assigned to the user device and the one or more shared devices.

19. The non-transitory, computer-readable storage medium of claim 15 further comprising determining infection registered at a public health data base associated with user device and possible user device exposures over a certain day.

20. The non-transitory, computer-readable storage medium of claim 15 further comprising logging proximity data received by the administrative service and sharing with a public health data base.
